(19) United States
(12) Patent Application Publication
(10) Pub. No.: US 2023/0022880 A1
(43) Pub. Date: Jan. 26, 2023

(54) Antimicrobial Composition

(71) Applicant: INSTITUTE OF TECHNOLOGY SLIGO, Sligo (IE)

(72) Inventors: James Joseph BRENNAN, Sligo (IE); Thomas Patrick PATTON, Sligo (IE); John Reginald BARRETT, Sligo (IE)

(21) Appl. No.: 17/783,889

(22) PCT Filed: Dec. 9, 2020
(86) PCT No.: PCT/EP2020/085376

§ 371 Date: Jun. 9, 2022

(30) Foreign Application Priority Data
Dec. 9, 2019 (GB) 1918021.5

(51) Int. Cl. A01N 59/00; A01N 59/08; A61K 33/40; A61K 33/14; A61K 31/37; A61K 47/26; A01P 1/00

(52) U.S. Cl. CPC A01N 59/00; A01N 59/08; A61K 33/40; A61K 33/14; A61K 31/37; A61K 47/26; A01P 1/00

(57) ABSTRACT

A composition comprising a hydrogen peroxide source and at least one metal halide. The hydrogen peroxide source comprises hydrogen peroxide and a means for generating hydrogen peroxide. The means for producing hydrogen peroxide comprises at least one oxidoreductase and at least one oxidoreductase substrate. The oxidoreductase substrate comprises at least one sugar, said sugar located within the composition. The composition is held under conditions that render the components inactive until rehydrated.

### FIELD OF THE INVENTION

The present invention relates to an antimicrobial composition and applications thereof. In particular, the invention relates to compositions comprising a hydrogen peroxide source.

### BACKGROUND TO THE INVENTION

Well-known antimicrobial compositions include conventional treatments such as antiseptics and antibiotics. Other treatments include silver-containing gels, compounds containing heavy metals and solutions of hydrogen peroxide and natural and synthetic pharmaceutically active substances. However, treatments such as antibiotics have disadvantages because of the emergence of antibiotic resistance. Furthermore, high levels of hydrogen peroxide have a toxic effect. In addition, hydrogen peroxide in solution is typically unstable and it is difficult to provide a sustained delivery system for this material. Thus, for a wide variety of different reasons, conventional antimicrobial treatments have many drawbacks.

Additionally, there are a number of naturally occurring antimicrobial systems known which rely on the ability of certain oxidising agents to disrupt metabolic processes of bacteria, fungi and viruses. For example, WO 03/090800 is directed to wound dressings comprising hydrated hydrogels and enzymes. Specifically, this patent describes the need to keep the enzyme substrate physically separated from the oxidoreductase enzyme prior to the use of the dressing. This prevents an unwarranted reaction which according to WO 03/090800 is undesirable. Thus, the wound dressing of WO 03/090800 can only function when it has been used or applied to a wound i.e. after it has been brought in contact with an appropriate enzyme substrate.

In recent years there has been a resurgence of interest in the therapeutic efficacy of honey, particularly in the area of wound healing. As a natural product, honey offers an attractive alternative to conventional treatments. Even though honey has been used for hundreds of years as a treatment for wounds, it is only relatively recently that the antibacterial properties of honey have been researched.

One example of a hydrogen peroxide based antimicrobial composition is A³IS of WO 2008/041218 A1, which comprises a range of sugars, water and the enzyme, glucose oxidase. Said composition provides a medium whereby a pool of hydrogen peroxide is stabilised for immediate use on application, followed by sustained release of hydrogen peroxide over a prolonged period.

In particular, WO 2008/041218 A1 describes A³IS as a storage-stable two-phase release formulation. The sugar, substrate, enzyme and water being further defined by % w/v.

Nail fungus, also called onychomycosis, is a common condition that starts as a white or yellow spot under the tip of the fingernail or toenail. Fungal nail infections are caused by various fungi, the most common being dermatophyte. Other causes of fungal nail infection include yeasts and moulds.

There remains a need for new and improved compositions with veterinary and animal husbandry applications.

Other applications include the treatment of microbial infections and the treatment or management of wound care and/or burns in animals, and Campylobacter infections in poultry.

There remains a need for new and improved antimicrobial compositions that overcome the above-mentioned disadvantages and can provide improved antibacterial activity.

### SUMMARY OF THE INVENTION

According to a first aspect of the present invention, there is provided a composition comprising a hydrogen peroxide source and at least one metal halide. The hydrogen peroxide source comprises hydrogen peroxide and a means for generating hydrogen peroxide. The means for producing hydrogen peroxide comprises at least one oxidoreductase and at least one oxidoreductase substrate. The oxidoreductase substrate comprises at least one sugar, said sugar located within the composition. The composition is held under conditions that render the components inactive until rehydrated.

The inventors have surprisingly found that the antimicrobial properties of compositions comprising a hydrogen peroxide source were greatly enhanced by the inclusion of a halide ion as was the ability of the material to generate a stable pool of hydrogen peroxide.

In another embodiment of the present invention the metal halide is selected from a metal chloride, metal fluoride, metal iodide, metal bromide and any mixtures thereof.

In a further embodiment of the present invention the metal chloride is selected from magnesium chloride, calcium chloride, potassium chloride, sodium chloride, lithium chloride, nickel chloride, silver chloride, ferric/ferrous chloride, potassium chloride, hydrogen chloride, copper chloride, chromium chloride manganese chloride, cobalt chloride, zinc chloride, barium chloride, beryllium chloride, cadmium chloride, aluminium chloride, gold chloride, titanium chloride and any ions, salts, isomers of mixtures thereof.

In a preferred embodiment the metal chloride is sodium chloride.

In another embodiment the composition further includes 8 methoxy psoralen.

In a further embodiment, the oxidoreductase is selected from one or more of the following: glucose oxidase, hexose oxidase, cholesterol oxidase, galactose oxidase, pyranose oxidase, choline oxidase, pyruvate oxidase, glycolate oxidase and/or amino acid oxidase.

According to a preferred embodiment of this aspect of the invention, the oxidoreductase is glucose oxidase.

In another embodiment of this aspect of the present invention, the oxidoreductase enzyme is present in the system at an activity of at least 10 U per 100 g of the system.

Generally speaking, one unit (U) is that amount of enzyme causing the oxidation of one micromole of glucose per minute at 25° C. and pH 7.0. It will be understood that there must be sufficient oxidoreductase present to catalyze the substrate to form hydrogen peroxide as needed.

In a preferred embodiment of this aspect of the present invention, the oxidoreductase is present in the system at an activity of at least 1000 per 100 g of the system.

In a more preferred embodiment of this aspect of the present invention the oxidoreductase is present in the system at an activity of at least 14000 per 100 g of the system.

In a still more preferred embodiment of this aspect of the present invention the oxidoreductase is present in the system at an activity of at least 5600 U per 100 g of the system.

In a most preferred embodiment of this aspect of the present invention the oxidoreductase is present in the system at an activity of at least 1250000 per 100 g of the system.

It will be understood that each oxidoreductase acts on a specific substrate. The corresponding substrates for each aforementioned oxidoreductase are D-glucose, hexose, cholesterol, D-galactose, pyranose, choline, pyruvate, glycolate and/or amino acid respectively.

It will be understood that a mixture of one or more oxidoreductase and one or more oxidoreductase substrates may be used.

In a further embodiment the oxidoreductase substrate is selected from one or more of D-glucose, hexose, cholesterol, D-galactose, pyranose choline, pyruvate, glycolate and/or amino acid.

In a preferred embodiment the oxidoreductase substrate is selected from one or more of D-glucose, hexose, D-galactose and/or pyranose. In a more preferred embodiment the oxidoreductase substrate is D-glucose.

In a further embodiment D-glucose is present up to 90% w/w. In a preferred embodiment D-glucose is present up to 85% w/w.

According to a preferred embodiment of the present invention, the oxidoreductase substrate is present from 20% to 85% w/w.

In another embodiment of the present invention, the composition forms a system, the system comprising a secondary oxidoreductase and a secondary oxidoreductase substrate.

One advantage of a secondary oxidoreductase: oxidoreductase substrate is that a tertiary source of hydrogen peroxide is provided by the composition.

Another advantage is that the secondary reaction further fuels the production of hydrogen peroxide by the composition, further prolonging the antimicrobial action of said composition, as exhaustion of the first oxidoreductase: oxidoreductase substrate is overcome.

In a further embodiment, the secondary oxidoreductase is selected from one or more of maltase, sucrase, sucrase-isomaltase, invertase, β-galactosidase, lactase, xanthine oxidoreductase and L-amino acid oxidase.

In a further embodiment the secondary oxidoreductase substrate is selected from one or more of maltose, sucrose, fructose, lactose, xanthine and L-amino acids.

It will be understood that a mixture of one or more secondary oxidoreductase and one or more secondary oxidoreductase substrates may be used.

In a further embodiment the secondary oxidoreductase substrate is located externally to the composition.

One advantage to the secondary substrate being located externally to the composition is that the secondary oxidoreductase: oxidoreductase substrate reaction cannot take place until the secondary oxidoreductase substrate, which drives the reaction, is present.

The external location may include, but is not limited to, milk, fruit juices, malted drinks, beer, fruits, vegetables, grains and grain-based products like breads and pastries. The external location may also include, for example, the mammary glands of an animal.

It will be appreciated that glucose will not be produced by this secondary oxidoreductase: oxidoreductase substrate pairing until, for example, the 6-galactosidase (i.e. the first part of this pairing, oxidoreductase) is in contact with the milk, which provides an external source of lactose (i.e. the second part of this pairing, oxidoreductase substrate).

Optionally, the system may comprise one or more sugars, which are in addition to any sugars that are an oxidoreductase substrate.

In one embodiment of this aspect of the invention, the one or more sugars may be selected from one or more of sucrose, fructose and/or maltose.

In a further embodiment of this aspect of the present invention the one or more sugars are present from 5% to 80% w/w.

In a preferred embodiment of this aspect of the present invention the one or more sugars are present from 5% to 70% w/w. In a more preferred embodiment of the present invention, the one or more sugars are present from 10% to 70% w/w.

In a further embodiment of this aspect of the invention, the one or more sugars are present in combination with the oxidoreductase substrate at a ratio of sugar to substrate of approximately 10:1 to 0.01:1.

In a preferred embodiment of this aspect of the invention, the one or more sugars are present in combination with the oxidoreductase substrate at a ratio of sugar to substrate of approximately from 3.5:1 to 0.05:1.

The preferred upper ratio of 3.5:1 is based on minimum oxidoreductase substrate content of 20%, and a maximum one or more sugar content of 70%. The preferred lower ratio of 0.05:1 is based on a maximum oxidoreductase substrate content of 85%, and one or more sugar content of 5%.

Ideally, the oxidoreductase substrate, preferably glucose or any other suitable substrate, and the one or more sugars are present in the system in the following ranges (based on the weight of the total system):

| Substrate for | Range |
|---|---|
| oxidoreductase enzyme | (% w/w) |
| Glucose | 10 to 85 |
| Additional Sugars | |
| Fructose | 8 to 50 |
| Maltose | 4 to 15 |
| Sucrose | 0.5 to 3 |

Ideally, the ratio of fructose:oxidoreductase substrate:maltose:sucrose is from approximately 1.5:4:1:0.1 to approximately 4.5:5:2:1.7. In a preferred embodiment the ratio is approximately 4.5:4:1:1.7. In a most preferred embodiment the ratio is approximately 4.5:4.1:1.2:0.2.

In another embodiment of the present invention the components hereinbefore described are in solution.

In a preferred embodiment of the present invention the solution is aqueous.

In another embodiment of the present invention the composition comprises a solvent.

In a preferred embodiment of the present invention the solvent is present from 10% to 20% by weight based on the weight of the total composition.

More preferably, solvent may be present a level from approximately 10% to approximately 15% by weight based on the weight of the total composition.

In a most preferred embodiment of the present invention the solvent is water.

The amount of solvent or water present in the composition initially is a crucial aspect of the invention. The addition of excess solvent/water can lead to instability in the composition, as excess solvent/water may give rise to hydrolysis of the glucose oxidase, so it is important that solvent/water is only initially present within defined parameters. In addition, the composition requires sufficient solvent/water to permit H₂O₂ release, ease of application and to prevent precipitation of sugars during storage.

One advantage of limiting the initial water content of the composition is that the composition is held under conditions that render the components inactive until rehydrated. Rehydration acts as the stimulus to kick-start the hydrolysis reaction.

In another embodiment of the present invention, the composition has a pH from approximately 3 to 8, preferably from 4 to 8, more preferably from 5 to 7, most preferably approximately 5.5.

The pH is important because it plays a critical role in many therapeutic aspects of the present invention, for example wound healing and also ensures that the oxidoreductase has the correct conditions for needed for optimal activity. For example, Manuka honey has a variable pH around 4. This pH is unsuitable for optimal oxidoreductase enzyme activity and would not be desirable when treating wounds. Thus, the ability to manipulate pH is highly desirable and a significant advantage of the present invention. Advantageously, the pH of the present system may be set at a pH as required for the particular application. Buffering agents may be used to manipulate the pH. Optionally, the system further comprises a buffering agent, preferably carbonic acid-bicarbonate and/or phosphoric acid/disodium hydrogen phosphate. Preferably, the buffering agent is pre-dissolved in and replaces part of the water of the system. Different concentrations of buffering agent can be used depending on the desired pH.

In a further embodiment of the present invention the hydrogen peroxide source is A³IS. The antimicrobial hydrogen peroxide producing composition, A³IS, is a storage-stable 2-phase release aqueous composition comprising: glucose oxidase with an activity of at least 10 U/100 g of the composition; D-glucose present from 20% to 85% w/v; one or more of sucrose, fructose and maltose present from 5% to 70% w/v combined; hydrogen peroxide; and, water present from 10% to 20% w/v. A³IS has a pH from approximately 3 to 8 and is characterised by the hydrogen peroxide release profile, wherein hydrogen peroxide is available for immediate release at a level of at least 0.1 mg/L followed by sustained release of hydrogen peroxide over a 24 hour period upon rehydration of the composition.

According to a second aspect of the present invention, there is provided a medicament comprising the aforementioned composition of the present invention, and a suitable delivery system.

In a further embodiment of the present invention, the delivery system is a topical delivery system suitable for topical administration of the composition as hereinbefore described.

In a preferred embodiment of the present invention, the topical delivery system is selected from plasters, dressings, woven spun materials, fibres, fabrics, hydrocolloids, masks, gels, creams, solutions, atomisable formulations, nebulisable formulations and any mixtures thereof.

In another embodiment of the present invention, the delivery system is an enteral delivery system suitable for oral administration of the composition as hereinbefore described.

In a preferred embodiment of the present invention the enteral delivery system is selected from one of solid dosage form and powdered dosage form.

In a further embodiment of the present invention, the delivery system is a parenteral delivery system suitable for injection administration of the composition as hereinbefore described.

In a third aspect of the present invention the aforementioned composition is suitable for use as a medicament.

In another embodiment of the present invention the aforementioned composition is suitable for use as an antimicrobial.

In another embodiment of the present invention, the aforementioned composition is suitable for use in the treatment or prophylaxis of fungal nail infection.

In another embodiment of the present invention, the aforementioned composition is suitable for use in the treatment or prophylaxis of Campylobacter infection.

In a preferred embodiment of the present invention the Campylobacter infection is in poultry.

In another embodiment of the present invention the aforementioned composition is suitable for use in the treatment or prophylaxis of Cryptosporidium infections.

In a preferred embodiment of the present invention the Cryptosporidium infection is in ruminants.

In a most preferred embodiment of the present invention the Cryptosporidium infection is in cattle.

Sodium chloride acts against catalyse which is a component in both vaginal fluid and semen so therefore the composition of the invention can be used as a medicament in the treatment of vaginosis and the preparation of a medicated condom.

In other embodiments of the invention, the composition can be used as a medicament in the treatment of wounds, infectious keratitis, collagen deficiency disorders, colony collapse disorder/pesticide detoxification in bees, methane reduction in ruminants, bacterial vaginosis, biofilm removal, mastitis, induction of hermetic effects and use as a preservative for foodstuffs.

### BRIEF DESCRIPTION OF THE DRAWINGS

The invention will be more clearly understood by the following description of some embodiments thereof, given by way of example only with reference to the accompanying drawings, in which:

FIG. 1 is graph showing catalase inhibition by sodium chloride;

FIG. 2 is a graph showing the effects of metal halides on GOX activity;

FIG. 3 is a bar chart showing percentage (%) performance improvement against various species of micro-organisms when A³IS is combined with sodium chloride; and,

FIG. 4 is a bar chart showing the effectiveness of A³IS and A³IS+sodium chloride against the most drug resistant pathogens as identified by WHO.

### DETAILED DESCRIPTION OF THE PREFERRED EMBODIMENTS

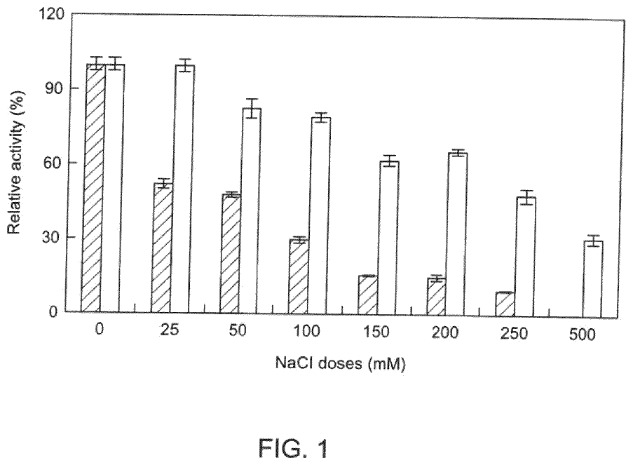

Referring to FIG. 1 the known effect of sodium chloride on catalase activity, changes in leaf catalase activity, from P. vulgaris (▪) and M. sativa (□), during in vitro incubation (1 h) in different NaCl doses is shown. Values, means of four independent assays, ±SE, are expressed as percentage relative to the activity without NaCl (P. vulgaris, 0.36 μkat mL⁻¹; M. sativa, 1.35 μkat mL⁻¹).

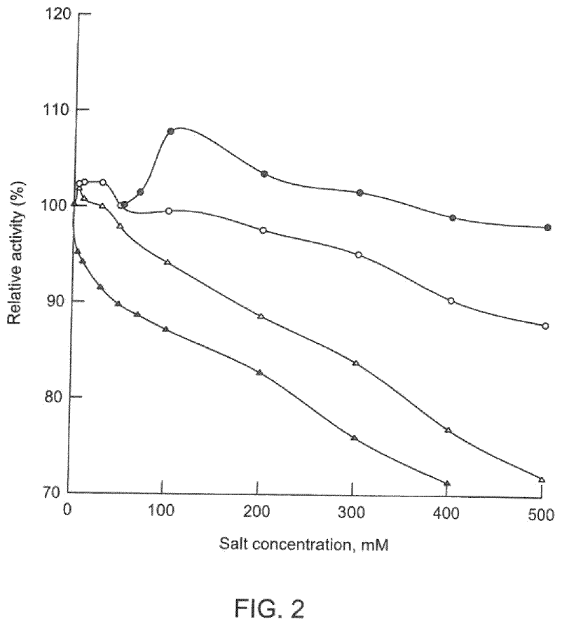

Referring now to FIG. 2 the known effects of metal chlorides on the GOX activity at pH 5.6. Sodium chloride, +; potassium chloride, ◯; calcium chloride, ∨; and magnesium chloride is shown.

The test formulations begin at 500 mM to 1000 mM where there is no evidence of GOX activation. The effect of halide ions on the activity of the glucose oxidase enzyme indicates that a significant reduction in the activity should be expected.

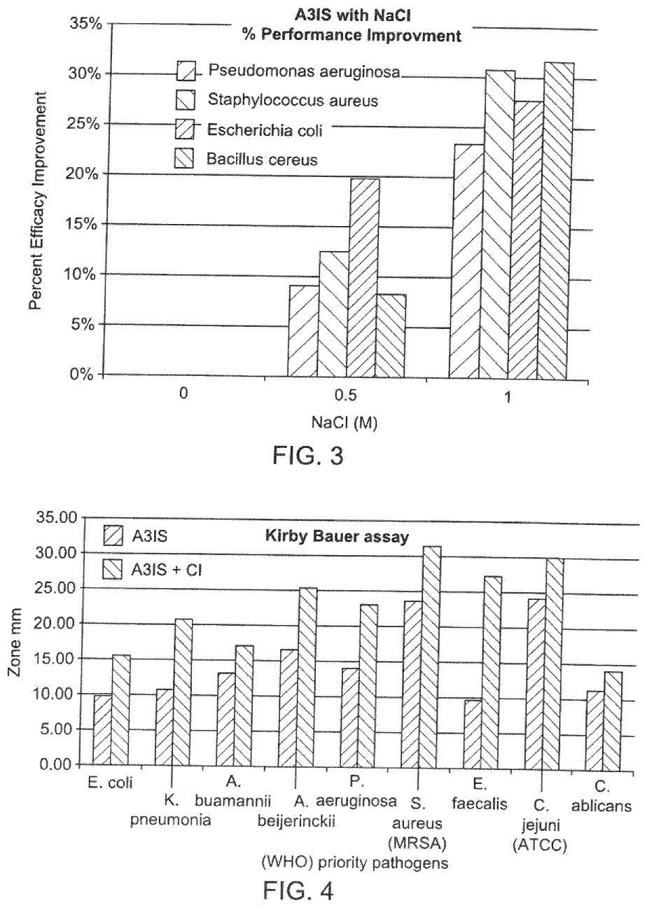

Referring now to FIG. 3 the combined effect of sodium chloride (NaCl) with A³IS against various species of microorganism is shown. Sodium chloride was added to the combination at 0.5M or 1M. The addition of NaCl saw a significant improvement of performance across all species of microorganisms tested against A³IS alone (control; 0M NaCl).

Referring now to FIG. 4 the effect of A³IS alone and in combination with sodium chloride against various drug resistant pathogens is shown.

### EXAMPLES

### Example 1: General Materials and Methods

Kirby-Bauer Antibiotic Testing

The purpose of the Kirby-Bauer disk diffusion susceptibility test is to determine the sensitivity or resistance of pathogenic aerobic and facultative anaerobic bacteria to various antimicrobial compounds.

TABLE 1

| | Drug Class | | | |
|---|---|---|---|---|
| presence | Antibiotic | WHO priority | | |
| of catalase | Concentration (ug/disk) | pathogen | | |
| + | E. coli | Critical | High | Medium |

| | |
|---|---|
| + | K. pneumonia |
| + | Acinetobacter beijerinckii |
| + | Acinetobacter baumannii |
| + | P. aeruginosa |
| + | S. aureus (MR) |
| − | E. faecalis |
| + | C. jejuni |
| + | Neisseria gonorrhoeae |

MR = methicilin resistant

Formulations comprising a hydrogen peroxide source in combination with various metal halides were prepared and subjected to a variety of tests to assess their physicochemical properties and also, to ensure that the antimicrobial characteristics of the material had not been compromised in the reformulation activity. The ability of the material to form a stable pool of hydrogen peroxide was also assessed.

Microbial Strains:

Escherichia coli (NCIMB 8545), Staphylococcus aureus (NCIMB 9518) and Pseudomonas aeruginosa (NCIMB 8626) are grown on nutrient agar or in nutrient broth for 24 hrs at 37° C.

Candida albicans (NCIMB 3179) and Saccharomyces cerevisiae are grown on sabaroud dextrose agar or in sabaroud dextrose broth for 24 hrs at 37° C. sabaroud dextrose agar or in sabaroud dextrose broth for 24 hrs at 37° C.

Bacterial growth is monitored by measuring the culture optical density (OD) in a spectrophotometer (Anthos 2010) at a wavelength of 620 nm.

Antimicrobial Efficacy Improvement

Three formulations of A³IS were prepared by the addition of NaCl to result in the following concentration of NaCl to the aqueous phase before this is used to dissolve the sugars and enzyme.

| NaCl (M) | 0 | 0.5 | 1 |
|---|---|---|---|
| A³IS (300 g) | grmms | grmms | grmms |
| Purified Water I | 30 | 30 | 30 |
| D-(−)-Fructose PhEur | 114 | 110 | 105 |
| D-(+)-Glucose PhEur | 105 | 101 | 97 |
| D-(+)-Maltose mono- | 30 | 30 | 30 |
| hydrate ≥95.0% | | | |
| Sucrose PhEur | 4.5 | 4.5 | 4.5 |
| Purified Water II | 15 | 15 | 15 |
| B-D-Glucose: | 1.5 | 1.5 | 1.5 |
| oxygen 1- | | | |
| oxidoreductase | | | |
| NaCl | 0 | 8.77 | 17.53 |
| Total | 300 | 301 | 301 |

The formulations were assessed for antimicrobial activity against several species of micro-organisms (several of which produce catalase; Candida spp, Pseudomonas spp, E. coli) using the well diffusion bioassay, results were obtained by measuring the zones of inhibition (n=3).

Well Diffusion Methods—for Measurement of Microbial Inhibition

Agar plates are inoculated by swabbing overnight culture onto the plate surface. Plates are allowed to stand at room temperature for 15 minutes before use. Wells 8.2 mm diameter are bored into the surface of the agar. A 180 μl sample is placed into each well. The samples diffuse into the agar around the well and are assayed for an ability to produce a zone of inhibition. Plates are incubated for 24, 48 or 72 hrs and zones of inhibition are measured using an Autodata automatic zone reader. The diameter of zones, including the diameter of the well (8.2 mm), is recorded. HYDROGEN PEROXIDE CONCENTRATIONS AT TIME ZERO

| | H₂O₂ Concentration |
|---|---|
| Formulation | (mg/l) Range |
| A³IS | >0.5 < 100 |
| A³IS + 0.5 M | >25 < 150 |
| NaCl | |
| A³IS + 1.0 M | >25 <= 200 |
| NaCl | |

The concentration of these materials in the final product will be in the following ranges:

| Material | Concentration Range |
|---|---|
| NaCl | 0.1-1.0 M |

Antimicrobial activity tests were also carried out on NaCl with no discernible activity being found.

It can therefore be concluded that there is a synergistic action between A³IS and the ingredients added from an antimicrobial activity standpoint.

The new improved formulation gives rise to a higher concentration of hydrogen peroxide via sustained release.

### Example 2: Effect of NaCl with A3IS

The composition comprising a metal halide (NaCl), hydrogen peroxide (A3IS) and a means for producing hydrogen peroxide (A3IS) was prepared according to the above-mentioned methods.

Different microbial strains were each plated out according to the above-mentioned methods.

The broad antimicrobial efficacy of this composition was then tested against each microbial strain according to the above-mentioned methods.

The results of this testing is shown in FIG. 3 and FIG. 4 and discussed above. A comparison with a control formulation is shown in FIG. 4 and also discussed above. The control is A3IS alone.

From the results it can be concluded that a synergistic action between A³IS and NaCl occurs that leads to an improved antimicrobial effect. This antimicrobial effect is shown across a broad range of microorganisms.

Formulation

The system of the present invention may be in many different physical forms, including but not limited to liquid preparations, solid or semi-solid preparations. In order to prepare solid or semi-solid formulations, the ingredients of the system should be manipulated to lower the water content and increase the content of the other components.

The system of the present invention may be in the form of a liquid preparation. Liquid preparations include but are not limited to a syrup, paste, spray, drop, ointments, creams, lotions, oils, liniments and/or gels. A typical gel includes an alcoholic gel such as isopropanol, ethanol, or propanol and/or a hydrogel.

Alternatively, the system of the present invention may be in the form of a solid or semi-solid preparation. Solid or semi-solid preparations include but are not limited to capsules, pellets, gel caps, hydrogels, pills, pillules and/or globules. Other means used for conventional drug-delivery can be adopted, for example, liposomal delivery may be contemplated.

According to a preferred embodiment of this aspect of the invention, there is provided a pharmaceutical composition comprising the system of the invention together with at least one pharmaceutically acceptable excipient or adjuvant.

According to another embodiment, there is provided a dressing comprising the system or pharmaceutical composition of the invention. Such dressings include gauzes, bandages, films, gels, foams—Lyofoam®, hydrocolloids—Granuflex®, alginates—Kaltostat® (Comvita), hydrogels—Intrasite Gel® and polysaccharide pastes, granules and beads.

According to a particular embodiment, the system may be present together with a wound-dressing matrix. Ideally, the ratio of the system to wound-dressing matrix may be approximately 1:1, although other ratios are contemplated. The wound-dressing matrix may be a collagen or collagen-GAG (glycosaminoglycan) matrix.

It will be understood that the system or pharmaceutical composition of the invention, may be present in many different adminstration forms. These forms include but are not limited to forms adapted for topical, enteral or parenteral administration.

Forms suitable for topical administration include a topical ointment, cream, lotion, oil, liniment, liquid and/or gel. For example, the system of the present invention may be applied epicutaneously, intranasally, via eye and/or ear drops. One particular embodiment of this aspect of the invention provides the system or pharmaceutical composition of the invention in a form adapted for intramammary administration. In this situation, the system or pharmaceutical composition of the invention may be adapted for delivery as part of a teat seal or intramammary depot delivered via the teat canal. Further compositions may be adapted as tissues, bandages or dressings. This is particularly advantageous for the treatment of infections such as mastitis and has both medical and veterinary applications.

Another form suitable for topical administration includes the system or pharmaceutical composition of the invention wherein the system or composition is in a form adapted for delivery via a dissolvable film strip or strips. In this situation the system of the present invention is soluble upon application.

Enteral administration includes, but is not limited to oral administration. Other enteral administration forms include suppositories and enemas. Forms suitable for oral administration include a capsule, pellet, gel cap, pill, pillule, globule, lozenge, dental floss, toothpaste, mouthwash, dissolvable film strips and/or adapted for delivery as part of a mouth guard. According to one embodiment of this aspect, the system or pharmaceutical composition is in a form suitable for controlled or sustained-release delivery. For example, the oral administration form may have an enteric coating to provide for controlled or sustained-release delivery. This sustained release aspect is important for the treatment of Campylobacter infections in poultry and the treatment of Cryptosporidium infections in cattle.

Parenteral/enteral administration forms include, but are not limited to injection. For example, the system may be adapted for injection by intramammary administration. This is particularly useful for the treatment of mastitis. Intramammary injection by this means involves injection directly into the teat canal using a tube or syringe with a nozzle of appropriate size, e.g. approx. 1.0 mm. Injection in this situation is directed into a body cavity or abscess.

The composition of the invention which includes 8 methoxy psoralen may be useful in treating psoriatic nail. The inclusion of this material together with exposure of the nails to UV-A light would be particularly beneficial.

Sodium chloride acts against catalyse which is a component in both vaginal fluid and semen so the composition of the invention can be used as a medicament in the treatment of bacterial vaginosis and in the preparation of a medicated condom.

The terms “comprise” and “include”, and any variations thereof required for grammatical reasons, are to be considered as interchangeable and accorded the widest possible interpretation.

The term “hydrogen peroxide source” will be understood to cover hydrogen peroxide itself and/or a means for generating hydrogen peroxide.

In the specification, it will be understood that the term “antimicrobial” or “antibacterial” are used interchangeably herein and cover biocidal or biostatic activity against various types of micro-organisms including but not limited to bacteria, fungi, viruses, yeasts, parasitic or pathogenic micro-organisms and/or moulds.

In the specification the term “by weight”, “percentage by weight” or “% w/w” refers to the weight of the final composition or system. These w/w values are interchangeable with w/v.

It will be understood that the components shown in any of the drawings are not necessarily drawn to scale, and, like parts shown in several drawings are designated the same reference numerals.

It will be further understood that features from any of the embodiments may be combined with alternative described embodiments, even if such a combination is not explicitly recited hereinbefore but would be understood to be technically feasible by the person skilled in the art.

The invention is not limited to the embodiments hereinbefore described which may be varied in both construction and detail within the scope of the appended claims.

1. A composition comprising:
a hydrogen peroxide source; and
at least one metal halide;
wherein the hydrogen peroxide source comprises
hydrogen peroxide; and
a means for producing hydrogen peroxide;
wherein the means for producing hydrogen peroxide comprises
at least one oxidoreductase; and
at least one oxidoreductase substrate
wherein the oxidoreductase substrate comprises
at least one sugar, said sugar located within the composition; and
wherein the composition is held under conditions that render the components inactive until rehydrated.

2. The composition according to claim 1 wherein the metal halide is selected from a metal chloride, metal fluoride, metal iodide, metal bromide and any mixtures thereof.

3. The composition according to claim 1 wherein the metal chloride is selected from magnesium chloride, calcium chloride, potassium chloride, sodium chloride, lithium chloride, nickel chloride, silver chloride, ferric/ferrous chloride, potassium chloride, hydrogen chloride, copper chloride, chromium chloride manganese chloride, cobalt chloride, zinc chloride, barium chloride, beryllium chloride, cadmium chloride, aluminium chloride, gold chloride, titanium chloride and any ions, salts, isomers or any mixtures thereof.

4. The composition according to claim 3 wherein the metal chloride is sodium chloride.

5. The composition according to claim 1 further includes 8-methoxy psoralen.

6. The composition according to claim 1, wherein the oxidoreductase substrate is D-Glucose.

7. The composition according to claim 1, wherein the hydrogen peroxide source is in solution.

8. The composition according to claim 7, wherein the solution is aqueous.

9. The composition according to claim 1, wherein the composition comprises a solvent.

10. The composition according to claim 9, wherein the solvent is water.

11. The composition according to claim 1, wherein the composition has a pH of from approximately 3 to 8.

12. The composition according to claim 1 wherein the hydrogen peroxide source comprises glucose oxidase, D-glucose, additional sugars selected from one or more of sucrose, fructose and/or maltose, and hydrogen peroxide in an aqueous/non aqueous solution;
wherein glucose oxidase is present at an activity of at least 10 U per 100 g of the composition;
D-glucose is present from 20 to 85% by weight based on the weight of the total composition;
additional sugars selected from one or more of sucrose, fructose and/or maltose are present from 5 to 70% by weight based on the weight of the total composition;
water or another solvent is present from 10 to 20% by weight based on the weight of the total composition;
the composition has a pH from approximately 3 to 8; and
wherein the composition provides a two-stage hydrogen peroxide release in which
(a) hydrogen peroxide is available for immediate release from the composition at a level of at least 0.1 mg per litre; and
(b) the sustained release of further hydrogen peroxide for at least a twenty-four hour period occurs upon rehydration of the composition.

13. A medicament comprising a composition according to claim 1.

14. The medicament according to claim 13, wherein the medicament comprises a topical delivery system suitable for topical administration of the composition according to claim 1.

15. The medicament according to claim 13 wherein the medicament comprises an enteral delivery system suitable for oral administration of the composition according to claim 1.

16. The medicament according to claim 13, wherein the medicament comprising a parenteral delivery system suitable for injection administration of the composition according to claim 1.

17. The composition according to claim 1 for use as a medicament.

18. The composition according to claim 17 for use as an antimicrobial.

19. The composition according to claim 17 for use in the treatment or prophylaxis of fungal nail.

20. The composition according to claim 17 for use in the treatment or prophylaxis of a Campylobacter infection.

21. The composition according to claim 17 for use in the treatment or prophylaxis of Cryptosporidium infections.

22. The composition according to claim 1 for use as a medicament in the treatment of wounds, infectious keratitis, collagen deficiency disorders, colony collapse disorder/pesticide detoxification in bees, methane reduction in ruminants, bacterial vaginosis, biofilm removal, mastitis, induction of hermetic effects and use as a preservative for foodstuffs.
